(19) United States
(12) Patent Application Publication
(10) Pub. No.: US 2010/0261765 A1
(43) Pub. Date: Oct. 14, 2010

(54) MINERALOCORTICOID RECEPTOR MODULATORS

(76) Inventors: Philip E. Brandish, North Wales, PA (US); Mark E. Fraley, North Wales, PA (US); James C. Hershey, Collegeville, PA (US); Justin T. Steen, Lansdale, PA (US)

Correspondence Address: MERCK, P O BOX 2000, RAHWAY, NJ, 07065-0907, US

(21) Appl. No.: 12/747,943

(22) PCT Filed: Dec. 10, 2008
(86) PCT No.: PCT/US08/13566

§ 371 Date: Jun. 14, 2010

Related U.S. Application Data
(60) Provisional application No. 61/007,640, filed on Dec. 14, 2007.

(51) Int. Cl. A61K 31/4418; C07D 211/90; C07D 211/84; A61P 13/12; A61P 9/12; A61P 9/10; A61P 27/06; A61P 15/00; A61P 25/22; A61P 3/10

(52) U.S. Cl. 514/350; 546/321; 514/356; 546/298

(57) ABSTRACT

The present invention relates to dihydropyridine mineralocorticoid receptor modulating compounds having the structure: and their use in treating cardiovascular events.

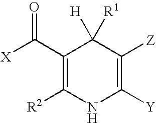

### FIELD OF THE INVENTION

The invention relates to novel mineralocorticoid receptor modulators of general formula (I). The invention also concerns related aspects, including processes for the preparation of the compounds, pharmaceutical compositions comprising one or more compounds of formula (I), in particular their use as mineralocorticoid receptor modulators in cardiovascular events and other pathologies.

### BACKGROUND OF THE INVENTION

The compounds described herein represent a novel structural class of mineralocorticoid receptor modulators.

Mineralocorticoids exert profound influences on a multitude of physiological functions by virtue of their diverse roles in growth, development and maintenance of homeostasis; these actions are mediated by the mineralocorticoid receptor (MR). In visceral tissues, such as the kidney and the gut, mineralocorticoid receptors regulate sodium retention, potassium excretion, and water balance in response to aldosterone. Elevations in aldosterone levels, or excess stimulation of mineralocorticoid receptors, are linked to several physiological disorders or pathologic disease states including Conn's Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, and disorders associated with excess catecholamine levels (Hadley, M. E., ENDOCRINOLOGY, 2^{nd} Ed., pp. 366-381, (1988); and Brilla et al., Journal of Molecular and Cellular Cardiology, 25(5), pp. 563-575 (1993)).

Additionally, elevated aldosterone levels have been increasingly implicated in congestive heart failure (CHF). In CHF, the failing heart triggers hormonal mechanisms in other organs in response to the attending reductions in blood flow and blood pressure seen with CHF. In particular, the kidney activates the renin-angiotensin-aldosterone system (RAAS) causing an increase in aldosterone production by the adrenals which, in turn, promotes water and sodium retention, potassium loss, and further edema. Although historically it was believed that aldosterone participated in the etiology of CHF only as a result of its salt retaining effects, several recent studies have implicated elevated aldosterone levels with events in extra-adrenal tissues and organs, such as myocardial and vascular fibrosis, direct vascular damage, and baroreceptor dysfunction. Pitt et al., New Eng. J. Med., 341:709-717 (1999). These findings are particularly significant since angiotensin converting enzyme (ACE) inhibitors, which were once thought to completely abolish aldosterone production, are now believed to only transiently suppress aldosterone production which has been shown to occur in extra-adrenal tissues, including the heart and vasculature. Weber, New Eng. J. Med., 341:753-755 (1999); Fardella and Miller, Annu. Rev. Nutr., 16:443-470 (1996).

Published results from RALES (Randomized Aldactone Evaluation Study) confirmed the involvement of aldosterone acting via MR in CHF (Pitt et al., New Eng. J. Med., 341:709-717 (1999)). It was demonstrated that the use of spironolactone, a well-known competitive MR antagonist, in combination with standard CHF therapy, reduced cardiac related mortality by 30% and frequency of hospitalization by 33% in patients suffering from advanced CHF. However, spironolactone therapy has also been associated with attending side effects such as gastric bleeding, diarrhea, azotemia, hyperchloremic metabolic acidosis and type-4 renal tubule acidosis, nausea, gynecomastia, erectile dysfunction, hyperkalemia, and irregular menses.

Thus, the mineralocorticoid receptor represents a viable target for CHF therapy either alone or in combination with conventional CHF therapies such as vasodilators (ACE inhibitors), inotropics (digoxin), diuretics, or beta blockers. Molecules, preferably non-steroids, which bind to the mineralocorticoid receptor and modulate receptor activity without the attending side effects current therapies would be particularly desirable.

Mineralocorticoid receptor antagonists have been approved for the treatment of hypertension and heart failure, but use of these generally well-tolerated drugs is limited due to mechanism-based hyperkalemia in some patients. To date, all approved modulators are full antagonists of the receptor and can cause a pathological increase in serum potassium concentration in some patients. This effect is increased in those patients also taking RAAS pathway blockers or those with impaired renal functioning and, as it is potentially lethal, requires monitoring by a specialist. There is accumulating evidence to suggest that the molecular and physiological mechanisms involved in efficacy and hyperkalemia are distinct. Because non-kalemic mineralocorticoid receptor modulators would clearly be safer than such current approved compounds, there is therefore a need for modulators of mineralocorticoid receptor function that are not hyperkalemic.

Hence, it would be desirable to develop a compound that would resolve efficacy from hyperkalemia by exploiting the unique opportunity offered by the nuclear receptor target class, i.e., to selectively modulate specific genes or pathways. It would be further desirable if the compounds exhibited dual activity; i.e., MR inhibition, useful in the treatment of such conditions as CHF and calcium channel antagonism, useful for treating hypertension.

### SUMMARY OF THE INVENTION

The present invention is directed to certain compounds and their use as mineralocorticoid receptor modulators, including treatment of conditions known to be associated with the mineralocorticoid receptor. The invention includes compounds of Formula I:

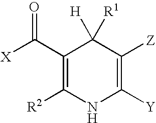

and pharmaceutically acceptable salts thereof, or an optical isomer thereof, wherein

X is selected from the group consisting of alkyl carboxylate; allyl carboxylate; aryl carboxylate; alkyl carboxyamide and aryl carboxamide;

Y is selected from the group consisting of alkyl and thioalkyl;

R¹ is unsubstituted or substituted aryl;

R² is alkyl; and

Z is either cyano or substituted or unsubstituted aliphatic carboxylate.

### DETAILED DESCRIPTION OF THE DISCLOSURE

The compounds of Formula I above, and pharmaceutically acceptable salts thereof, are mineralocorticoid receptor modulators. The compounds are useful for modulating the mineralocorticoid receptor and treating conditions such as hypertension.

In one embodiment, X is selected from the group consisting of ethyl carboxylate; methyl carboxylate; benzyl carboxylate; allyl carboxylate; methoxyphenylcarboxamide; and ethoxyphenylcarboxamide.

In another embodiment, Y is selected from the group consisting of methyl; methylthiolate; ethylthiolate; and propylthiolate.

In yet another embodiment, R¹ is selected from the group consisting of chlorophenyl; dichlorophenyl; nitrophenyl; hydroxynitrophenyl; naphthyl; vinylphenyl; hydroxymethoxyphenyl and hydroxyethoxyphenyl.

In another embodiment, R² is methyl.

In another embodiment, Z is selected from the group consisting of carboxylate; cyano; benzyl carboxylate; allyl carboxylate; and isopropyl carboxylate.

Specific examples of compounds of formula I, and pharmaceutically acceptable salts thereof, include the following: diethyl 2,6-dimethyl-4-(4-nitrophenyl)-1,4-dihydropyridine-3,5-dicarboxylate; dimethyl 2,6-dimethyl-4-(1-naphthyl)-1,4-dihydropyridine-3,5-dicarboxylate; dimethyl 4-(2,3-dichlorophenyl)-2,6-dimethyl-1,4-dihydropyridine-3,5-dicarboxylate; diethyl 4-(2-hydroxy-3-nitrophenyl)-2,6-dimethyl-1,4-dihydropyridine-3,5-dicarboxylate; dimethyl 2,6-dimethyl-4-(2-vinylphenyl)-1,4-dihydropyridine-3,5-dicarboxylate; 4-(2-chlorophenyl)-5-cyano-6-(ethylthio)-N-(2-methoxyphenyl)-2-methyl-1,4-dihydropyridine-3-carboxamide; 4-(2-chlorophenyl)-5-cyano-N-(2-methoxyphenyl)-2-methyl-6-(methylthio)-1,4-dihydropyridine-3-carboxamide; dimethyl 4-(3,4-dichlorophenyl)-2,6-dimethyl-1,4-dihydropyridine-3,5-dicarboxylate; diethyl 2,6-dimethyl-4-(1-naphthyl)-1,4-dihydropyridine-3,5-dicarboxylate; dibenzyl 4-(4-hydroxy-3-methoxyphenyl)-2,6-dimethyl-1,4-dihydropyridine-3,5-dicarboxylate; 4-(2-chlorophenyl)-5-cyano-N-(2-methoxyphenyl)-2-methyl-6-(propylthio)-1,4-dihydropyridine-3-carboxamide; diallyl 2,6-dimethyl-4-(3-nitrophenyl)-1,4-dihydropyridine-3,5-dicarboxylate; 5-cyano-4-(2-ethoxyphenyl)-2-methyl-6-(methylthio)-N-phenyl-1,4-dihydropyridine-3-carboxamide; ethyl 4-(2-chlorophenyl)-5-cyano-6-(methylthio)-2-propyl-1,4-dihydropyridine-3-carboxylate; ethyl isopropyl 2,6-dimethyl-4-(4-nitrophenyl)-1,4-dihydropyridine-3,5-dicarboxylate.

The present invention also encompasses a pharmaceutical formulation comprising a pharmaceutically acceptable carrier and the compound of Formula I or a pharmaceutically acceptable crystal form or hydrate thereof.

Compounds of formula (I) or the above-mentioned pharmaceutical compositions are also of use in combination with other pharmacologically active compounds such as antihypertensive or antiinflammatory compounds including ACE-inhibitors, neutral endopeptidase inhibitors, angiotensin II receptor antagonists, renin inhibitors, endothelin receptors antagonists, vasodilators, calcium channel antagonists, potassium activators, diuretics, sympatholitics, beta-adrenergic antagonists, alpha-adrenergic antagonists, other mineralocorticoid receptor modulators, glucocorticoids, glucocorticoid receptor modulators, estrogen receptor modulators, and androgen receptor modulators and other active compounds commonly administered with antihypertensives to treat diseases associated with hypertension, organ damage and inflammation, including, but not limited to cholesterol reducing statins, cholesterol absorption inhibitors or with other drugs beneficial for the prevention or the treatment of the above-mentioned diseases.

The term “alkyl” shall mean straight or branched chain alkanes of one to ten total carbon atoms, or any number within this range (i.e., methyl, ethyl, 1-propyl, 2-propyl, n-butyl, s-butyl, t-butyl, etc.).

The term “aryl” as used herein, except where otherwise specifically defined, refers to unsubstituted, mono- or poly-substituted aromatic groups such as phenyl or naphthyl.

Unless otherwise specifically noted as only “unsubstituted” or only “substituted”, defined groups are unsubstituted or substituted. Preferably, substituents are selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl)S(O)₀₋₂—, aryl-S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆alkyl)O(C₁-C₆ alkyl)-, (C₀-C₆ alkyl)C(O)₁₋₂(C₀-C₆ alkyl)-, (C₀-C₆ alkyl)OC(O)NH—, aryl, aralkyl, heteroaryl, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl. The term “substituted” is understood to include mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., aryl, a heteroaromatic ring, or a saturated heterocyclic ring) provided such ring substitution is chemically allowed and results in a stable compound.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention.

Pharmaceutically acceptable salts include both the metallic (inorganic) salts and organic salts; a list of which is given in Remington's Pharmaceutical Sciences, 17th Edition, pg. 1418 (1985). It is well known to one skilled in the art that an appropriate salt form is chosen based on physical and chemical properties. As will be understood by those skilled in the art, pharmaceutically acceptable salts include, but are not limited to salts of inorganic acids such as hydrochloride, sulfate, phosphate, diphosphate, hydrobromide, and nitrate or salts of an organic acid such as malate, maleate, fumarate, tartrate, succinate, citrate, acetate, lactate, methanesulfonate, p-toluenesulfonate or palmoate, salicylate and stearate. Similarly pharmaceutically acceptable cations include, but are not limited to sodium, potassium, calcium, aluminum, lithium and ammonium (especially ammonium salts with secondary amines). Preferred salts of this invention for the reasons cited above include potassium, sodium, calcium and ammonium salts. Also included within the scope of this invention are crystal forms, hydrates and solvates of the compounds of Formula I.

The compounds of Formula I can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Certain of the compounds employed in the present invention may carry an acidic moiety (e.g., —COOH or a phenolic group), in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The present invention is further directed to a method of treating a condition in a subject in need thereof Such a condition may be selected from those conditions such as hypertension, congestive heart failure, pulmonary hypertension, systolic hypertension, renal insufficiency, renal ischemia, renal failure, renal fibrosis, cardiac insufficiency, cardiac hypertrophy, cardiac fibrosis, myocardial ischemia, vascular inflammation, vascular dementia, cardiomyopathy, glomerulonephritis, renal colic, complications resulting from diabetes such as nephropathy, vasculopathy and neuropathy, macular degenerative disorders, metabolic syndrome, glaucoma, elevated intra-ocular pressure, atherosclerosis, post-angioplasty restenosis, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, wherein said method comprises the step of administering a compound as defined above to subject such as a human being or animal.

Embodiments of the method of the present invention include those in which the compound of Formula I administered to the subject is as defined in the compound embodiments, classes and sub-classes set forth above.

In another embodiment, the invention further relates to a method for the treatment and/or prophylaxis of diseases which are related to hypertension, congestive heart failure, pulmonary hypertension, macular degenerative disorders, metabolic syndrome, intraocular pressure, glaucoma, atherosclerosis, metabolic syndrome, and complications resulting from diabetes such as nephropathy, vasculopathy and neuropathy.

The invention also relates to the use of compounds of formula (I) for the preparation of a medicament for the treatment and/or prophylaxis of the above-mentioned diseases.

The term “administration” and variants thereof (e.g., “administering” a compound) in reference to a compound of Formula I mean providing the compound or a prodrug of the compound to the individual in need of treatment or prophylaxis. When a compound of the invention or a prodrug thereof is provided in combination with one or more other active agents (e.g., an agent such as an angiotensin II receptor antagonist, renin inhibitor, ACE inhibitor, or other active agent which is known to reduce blood pressure), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients in the specified amounts, as well as any product which results, directly or indirectly, from combining the specified ingredients in the specified amounts.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit renin and thereby elicit the response being sought (i.e., an “inhibition effective amount”). When the active compound (i.e., active ingredient) is administered as the salt, references to the amount of active ingredient are to the free form (i.e., the non-salt form) of the compound.

In a preferred embodiment, this amount is comprised between 1 mg and 1000 mg per day. In a particularly preferred embodiment, this amount is comprised between 1 mg and 500 mg per day. In a more particularly preferred embodiment, this amount is comprised between 1 mg and 200 mg per day.

In the method of the present invention, the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

Compounds of the present invention can be made by a variety of methods depicted in the illustrative synthetic reaction scheme as shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2.sup.nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes and examples are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction scheme can be isolated and purified if desired using conventional techniques, including but not limited to, filtration, distillation, crystallization, chromatography, and the like. Such materials can be characterized using conventional means, including physical constants and spectral data.

Unless specifically stated otherwise, the experimental procedures were performed under the following conditions. Evaporation of solvent was carried out using a rotary evaporator under reduced pressure (600-4000 pascals: 4.5-30 mm Hg) with a bath temperature of up to 60° C. Reactions are typically run under nitrogen atmosphere at ambient temperature if not otherwise mentioned. Anhydrous solvent such as THF, DMF, Et₂O, DME and Toluene are commercial grade. Reagents are commercial grade and were used without further purification. Flash chromatography is run on silica gel (230-400 mesh). The course of the reaction was followed by either thin layer chromatography (TLC) or nuclear magnetic resonance (NMR) spectrometry and reaction times given are for illustration only. The structure and purity of all final products were ascertained by TLC, mass spectrometry, ¹H NMR and high-pressure liquid chromatography (HPLC). Chemical symbols have their usual meanings. The following abbreviations have also been used: v (volume), w (weight), b.p. (boiling point), m.p. (melting point), L (liter(s)), mL (milliliter(s)), g (gram(s)), mg (milligram(s)), mol (mole(s)), mmol (millimole(s)), eq. (equivalent(s)). Unless otherwise specified, all variables mentioned below have the meanings as provided above.

As shown in Reaction Scheme I, with specific reference to Compound 1-2 listed in Table 1, 1,4-dihydropyridines of the present invention can be prepared by the Hantzsch pyridine synthesis (Phillips, A. P. J. Am. Chem. Soc 1949, 71, 4003-4007). Accordingly, substituted aromatic aldehydes can be cyclized with methyl acetoacetate and aqueous ammonium hydroxide in alcohol with heating to provide 1,4-dihydropyridines.

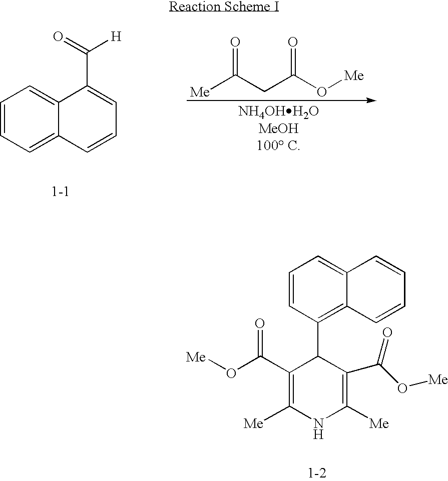

### Example 1

### Dimethyl 2,6-dimethyl-4-(1-naphthyl)-1,4-dihydropyridine-3,5-dicarboxylate (1-2)

A solution of 1-naphthaldehyde (1-1) (29 g, 190 mmol, 1.0 eq) and methyl acetoacetate (47 g, 400 mmol, 2.2 eq) in methanol (50 ml) and aqueous ammonium hydroxide (20 ml) was allowed to stand at room temperature for 1 hour and then heated at 100° C. for 16 hours. After cooling, the orange precipitate was filtered and washed with methanol resulting in yellow crystals. ¹H NMR (500 MHz, DMSO-d6) δ 8.89 (s, 1H), 8.48 (d, 1H, J=8.5 Hz), 7.81 (d, 1H, J=8.5 Hz), 7.67 (m, 1H), 7.52 (m, 1H), 7.43-7.39 (m, 3H), 5.69 (s, 1H), 3.33 (s, 6H), 2.28 (s, 6H). LRMS m/z (M+H) 352.1 found, 352.2 required.

The following compounds were prepared by simple modifications of the above procedures. Mineralocorticoid receptor binding affinity Ki values (nM) and FLIPR data are shown after the compound name.

TABLE 1

| | | | | | FLIPRIETRA Cav1.2 % Inhibition | | | | |
|---|---|---|---|---|---|---|---|---|---|
| | | MRBIND Ki (nM) | | | 0.3 | 1 | 3 | 10 | 30 |
| STRUCTURE | Trivial name | Rep#1 | Rep#2 | Average | μM | μm | μm | μm | μm |
| 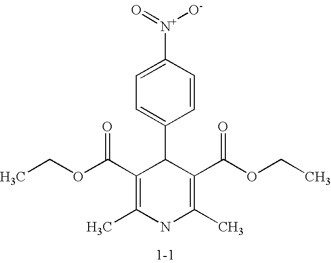 | diethyl 2,6- dimethyl-4-(4- nitrophenyl)-1,4- dihydropyridine- 3,5-dicarboxylate | 184 | 254 | 219 | 25 | 43 | 59 | 76 | |
| 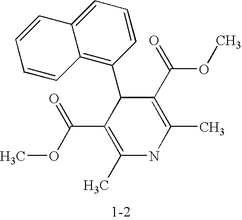 | Dimethyl 2,6- dimethyl-4-(1- naphthyl)-1,4- dihydropyridine- 3,5- dicarboxylate | 69 | 53 | 61 | 80 | 88 | 94 | 100 | 104 |
| 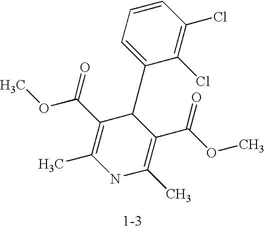 | dimethyl 4-(2,3- dichlorophenyl)- 2,6-dimethyl- 1,4- dihydropyridine- 3,5- dicarboxylate | 382 | 368 | 375 | 73 | 75 | 77 | 78 | 84 |
| 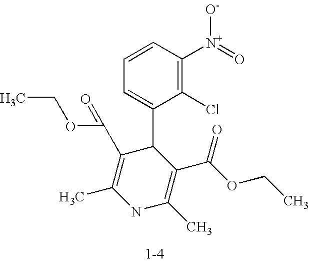 | diethyl 4-(2- hydroxy-3- nitrophenyl)-2,6- dimethyl-1,4- dihydropyridine- 3,5- dicarboxylate | 290 | 210 | 250 | 87 | 88 | 91 | 95 | |
| 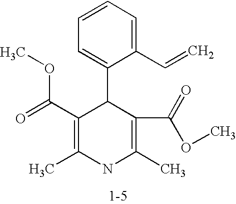 | dimethyl 2,6- dimethyl-4-(2- vinylphenyl)- 1,4- dihydropyridine- 3,5- dicarboxylate | 570 | 408 | 489 | 74 | 74 | 78 | 87 | |
| 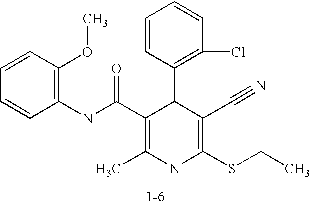 | 4-(2- chlorophenyl)-5- cyano-6- (ethylthio)-N-(2- methoxyphenyl)- 2-methyl-1,4- dihydropyridine- 3-carboxamide | 90 | 16 | 53 | 41 | 83 | 92 | 98 | 104 |
| 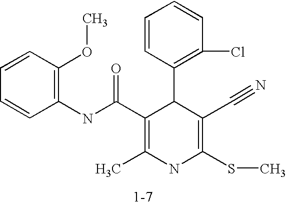 | 4-(2- chlorophenyl)-5- cyano-N-(2- methoxyphenyl)- 2-methyl-6- (methylthio)-1,4- dihydropyridine- 3-carboxamide | 250 | 137 | 193.5 | −3 | 14 | 35 | 74 | |
| 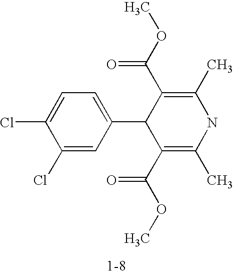 | dimethyl 4-(3,4- dichlorophenyl)- 2,6-dimethyl- 1,4- dihydropyridine- 3,5- dicarboxylate | 83 | 324 | 203.5 | 34 | 59 | 77 | 87 | 89 |
| 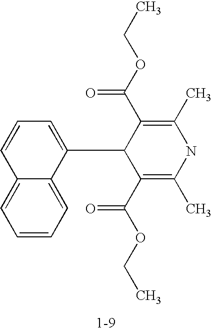 | diethyl 2,6- dimethyl-4-(1- naphthyl)-1,4- dihydropyridine- 3,5- dicarboxylate | 161 | 101 | 131 | 82 | 85 | 82 | 85 | 85 |
| 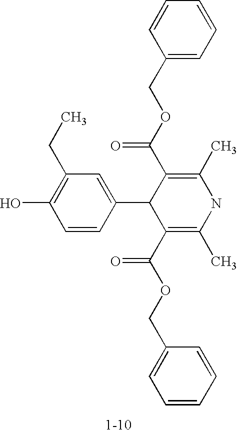 | dibenzyl 4-(4- hydroxy-3- methoxyphenyl)- 2,6-dimethyl- 1,4- dihydropyridine- 3,5- dicarboxylate | 32 | 11 | 21.5 | 45 | 76 | 87 | 93 | 106 |
| 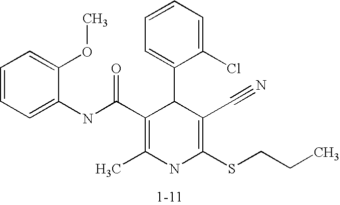 | 4-(2- chlorophenyl)-5- cyano-N-(2- methoxyphenyl)- 2-methyl-6- (propylthio)-1,4- dihydropyridine- 3-carboxamide | 11.6 | 18.2 | 14.9 | 31 | 69 | 90 | 96 | 102 |
| 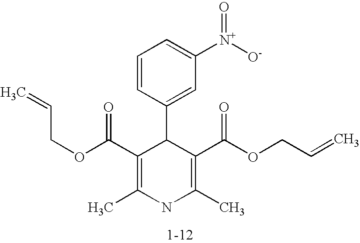 | diallyl 2,6- dimethyl-4-(3- nitrophenyl)-1,4- dihydropyridine- 3,5- dicarboxylate | 472 | 453 | 462.5 | 81 | 83 | 82 | 81 | |
| 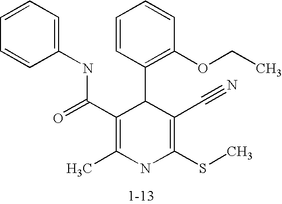 | 5-cyano-4-(2- ethoxyphenyl)-2- methyl-6- (methylthio)-N- phenyl-1,4- dihydropyridine- 3-carboxamide | 1138 | 1648 | 1393 | 4 | 26 | 55 | 82 | |
| 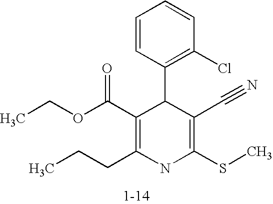 | ethyl 4-(2- chlorophenyl)-5- cyano-6- (methylthio)-2- propyl-1,4- dihydropyridine- 3-carboxylate | 23.6 | 51 | 37.3 | 53 | 73 | 74 | 80 | |
| 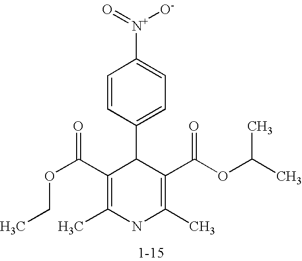 | ethyl isopropyl 2,6-dimethyl-4- (4-nitrophenyl)- 1,4- dihydropyridine- 3,5- dicarboxylate | 77 | 152 | 114.5 | 28 | 49 | 63 | 73 | 86 |

The following serves only to illustrate the invention and its practice and is not to be construed as a limitation on the scope or spirit of the invention.

### Measurement of Mineralocorticoid Receptor Binding Affinity

The binding affinity of compounds for the mineralocorticoid receptor was determined by measuring their ability to prevent binding of radiolabeled aldosterone to recombinant rhesus mineralocorticoid receptor in a traditional filter binding assay protocol.

Rhesus mineralocorticoid receptor cDNA was cloned from a cDNA library using and used to prepare a recombinant baculovirus encoding the rhesus mineralocorticoid receptor coding sequence by standard molecular biological and cell biological methods. Insect cells grown in culture were infected with the recombinant baculovirus and this resulted in the expression of recombinant rhesus mineralocorticoid receptor in those cells. Cells were collected and lysed. The lysates were clarified by centrifugation and stored at −80 C until use in the radioligand binding assay.

The assays were carried out in 20 mM Hepes, 10 mM Na₂MoO₄, 10 mM 2-mercaptoethanol, 157 mM sucrose, and 3.7 mM CHAPS. [³H]-Aldosterone (1 mCi/ml, 70-100 Ci/mmol) was purchased from Perkin Elmer (NET419). Test compounds were dissolved in DMSO and diluted in DMSO to 50 times the desired final concentrations for 3-fold serial dilution dose response curves. A working stock solution of [³H]-aldosterone was prepared by dilution of the commercial stock to 0.083 μM in assay buffer. The insect cell lysate containing rhesus mineralocorticoid receptor was thawed and diluted to 0.7 mg protein/mL. Assay were started by combining 20 μL of test compound solution, 920 μL of diluted insect cell lysate, and 60 μL of [³H]-aldosterone working solution in 2-mL 96-well polypropylene square well plates (USA Scientific) at 20° C. The mixture was incubated for 3 hr with continuous agitation on a platform shaker. The mixture was then filtered through 96-well GF/B filter plates (Packard) that had been previously treated with a solution of polyethylenimine (Sigma, P-3143). The filter plate was washed 3 times with 0.5 mL of 50 mM Tris-HCl, pH 7.4 and then dried overnight at 37° C. in a vacuum oven. The bottom of the plate was sealed and 40 μL of Microscint-20 (Packard, 6013621) was added to each well before counting radioactivity with a Topcount plate reader. Non-specific radioligand binding was determined by adding non-radiolabeled aldosterone (0.5 mM in DMSO) to the assay mixture to a final concentration of 10 μM in place of test compound. IC₅₀ and Ki values were determined using a four parameter logistic fit using a customized assay data analyzer software package.

Examples were tested in the ligand binding assay and demonstrated IC₅₀s less than 10,000 nM.

### Measurement of Calcium Channel Block

A high-throughput fluorescence assay for state-dependent block of L-type channels was established as a counterscreen for blockers of N-type calcium channels. A HEK293 cell line (Xia, et al., 2004) expressing L-type calcium channels, composed of 3 calcium channel subunits, Cav1.2 (alpha_{1C}), α₂-delta, beta₂ₐ, and an inwardly-rectifying potassium channel, Kir_{2.3} was used to develop a fluorescent high-throughput assay for L-type calcium channels. Expression of Kir2.3 in the cells ensures that the cell membrane potential can be reliably controlled by external potassium concentration (Xia, et al., 2004). This allows the cell membrane potential to be preset during compound incubation, which can be used to assay state-dependent channel block. Running the assay in the presence of high potassium (25 mM) sets the membrane potential to a value (−35 mV) at which approximately half of the calcium channels are inactivated and some are open. This condition favors block by many L-type calcium channel blockers that lower blood pressure, including dihydropyridines, phenylalkylamines and benzothiazepines.

Initially, the compounds were incubated in 0.005 μM calcium and 25 mM potassium for 30 minutes. Calcium influx was triggered by buffer addition that raises the calcium concentration to 2 mM while maintaining the potassium concentration at 25 mM. Changes in intracellular calcium were then monitored using a calcium-sensitive fluorescent dye (fluo-4) and a FLIPR^{TETRA} plate reader.

The following experimental protocol was used:

1. Cells were seeded in a Poly-D-Lysine Coated 384-well plate (50 μl/well) and incubated overnight at 37° C. under 10% CO₂;

2. Tissue culture media was removed and cells were washed with 0.06 ml 5.8 mM K Potassium Pre-polarization Buffer (PPB), which is 146.2 mM NaCl, 5.8 mM KCl, 0.005 mM CaCl2, 1.7 mM MgCl2, 10 HEPES, pH=7.2;

3. 0.04 ml of 4 μM fluo-4 (Molecular Probes; F-14202) and 0.02% Pluronic acid (Molecular Probes; P-3000) prepared in 5.8 mM K PPB supplemented with 10 mM Glucose was added to the cells;

4. Cells were then incubated in the dark at 25° C. for 30 minutes;

5. The dye was removed and cells were washed with 0.06 ml of 25 mM K Potassium Pre-polarization Buffer (PPB), which is 127 mM NaCl. 25 mM KCl, 0.005 mM CaCl₂, 1.7 mM MgCl₂, 10 HEPES, pH=7.2;

6. 0.025 ml of 25 mM K PPB was then added, with or without the presence of a test compound;

7. Cells incubated in the dark at 25° C. for 30 min

8. Cell plates are then read on the FLIPR^{TETRA} instrument, Excitation=480 nm, Emission=535 nm;

9. With FLIPR^{TETRA} continuously reading, 0.025 ml of Ca Trigger Buffer (CTB) is added, which is 119 mM NaCl, 25mM KCl, 4 mM CaCl₂, 1.7 mM MgCl₂, 10 HEPES, pH=7.2, and which was 2× the final assay concentration, to the cell plate.

The fluorescent L-type calcium channel assay, configured as described, is robust with an adequate signal to noise ratio, and is run in a 384 well format, allowing medium-to-high throughput testing of compounds. Values for the compounds of the present invention are presented in Table 1.

While the foregoing specification teaches the principles of the present invention, with examples provided for the purpose of illustration, it will be understood that the practice of the invention encompasses all of the usual variations, adaptations, or modifications, as fall within the scope of the following claims and its equivalents.

What is claimed is:

1. A compound of formula I, 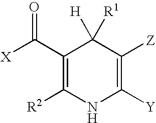
and pharmaceutically acceptable salts thereof, or an optical isomer thereof, wherein
X is selected from the group consisting of alkyl carboxylate; allyl carboxylate; aryl carboxylate; alkyl carboxyamide and aryl carboxamide;
Y is selected from the group consisting of alkyl and thioalkyl;
R¹ is unsubstituted or substituted aryl;
R² is alkyl;
Z is either cyano or substituted or unsubstituted aliphatic carboxylate.

2. A compound of claim 1, or a pharmaceutically acceptable salt thereof, wherein X is selected from the group consisting of ethyl carboxylate; methyl carboxylate; benzyl carboxylate; allyl carboxylate; methoxyphenylcarboxamide; and ethoxyphenylcarboxamide.

3. A compound of claim 1, wherein Y is selected from the group consisting of methyl; methylthiolate; ethylthiolate; and propylthiolate.

4. A compound of claim 1, wherein R¹ is selected from the group consisting of chlorophenyl; dichlorophenyl; nitrophenyl; hydroxynitrophenyl; naphthyl; vinylphenyl; hydroxymethoxyphenyl and hydroxyethoxyphenyl.

5. A compound of claim 1, wherein R² is methyl.

6. A compound of claim 1, wherein Z is selected from the group consisting of carboxylate; cyano; benzyl carboxylate; allyl carboxylate; and isopropyl carboxylate.

7. A compound of claim 1, or a pharmaceutically acceptable salt thereof, selected from the group consisting of diethyl 2,6-dimethyl-4-(4-nitrophenyl)-1,4-dihydropyridine-3,5-dicarboxylate; dimethyl 2,6-dimethyl-4-(1-naphthyl)-1,4-dihydropyridine-3,5-dicarboxylate; dimethyl 4-(2,3-dichlorophenyl)-2,6-dimethyl-1,4-dihydropyridine-3,5-dicarboxylate; diethyl 4-(2-hydroxy-3-nitrophenyl)-2,6-dimethyl-1,4-dihydropyridine-3,5-dicarboxylate; dimethyl 2,6-dimethyl-4-(2-vinylphenyl)-1,4-dihydropyridine-3,5-dicarboxylate; 4-(2-chlorophenyl)-5-cyano-6-(ethylthio)-N-(2-methoxyphenyl)-2-methyl-1,4-dihydropyridine-3-carboxamide; 4-(2-chlorophenyl)-5-cyano-N-(2-methoxyphenyl)-2-methyl-6-(methylthio)-1,4-dihydropyridine-3-carboxamide; dimethyl 4-(3,4-dichlorophenyl)-2,6-dimethyl-1,4-dihydropyridine-3,5-dicarboxylate; diethyl 2,6-dimethyl-4-(1-naphthyl)-1,4-dihydropyridine-3,5-dicarboxylate; dibenzyl 4-(4-hydroxy-3-methoxyphenyl)-2,6-dimethyl-1,4-dihydropyridine-3,5-dicarboxylate; 4-(2-chlorophenyl)-5-cyano-N-(2-methoxyphenyl)-2-methyl-6-(propylthio)-1,4-dihydropyridine-3-carboxamide; diallyl 2,6-dimethyl-4-(3-nitrophenyl)-1,4-dihydropyridine-3,5-dicarboxylate; 5-cyano-4-(2-ethoxyphenyl)-2-methyl-6-(methylthio)-N-phenyl-1,4-dihydropyridine-3-carboxamide; ethyl 4-(2-chlorophenyl)-5-cyano-6-(methylthio)-2-propyl-1,4-dihydropyridine-3-carboxylate; and ethyl isopropyl 2,6-dimethyl-4-(4-nitrophenyl)-1,4-dihydropyridine-3,5-dicarboxylate.

8. A pharmaceutical composition comprising an effective amount of a compound according to claim 1, or a pharmaceutically acceptable salt thereof, and a pharmaceutically acceptable carrier.

9. Use of a compound according to claim 1, or a composition according to claim 8, for the manufacture of a medicament for the treatment or prophylaxis of diseases which are related to hypertension, congestive heart failure, pulmonary hypertension, systolic hypertension, renal insufficiency, renal ischemia, renal failure, renal fibrosis, cardiac insufficiency, cardiac hypertrophy, cardiac fibrosis, myocardial ischemia, vascular inflammation, vascular dementia, cardiomyopathy, glomerulonephritis, renal colic, complications resulting from diabetes such as nephropathy, vasculopathy and neuropathy, macular degenerative disorders, metabolic syndrome, glaucoma, elevated intra-ocular pressure, atherosclerosis, post-angioplasty restenosis, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

10. A method for the treatment or prophylaxis of diseases which are related to hypertension, congestive heart failure, pulmonary hypertension, systolic hypertension, renal insufficiency, renal ischemia, renal failure, renal fibrosis, cardiac insufficiency, cardiac hypertrophy, cardiac fibrosis, myocardial ischemia, vascular inflammation, vascular dementia, cardiomyopathy, glomerulonephritis, renal colic, complications resulting from diabetes such as nephropathy, vasculopathy and neuropathy, macular degenerative disorders, metabolic syndrome, glaucoma, elevated intra-ocular pressure, atherosclerosis, post-angioplasty restenosis, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal, comprising the administration to a patient of a pharmaceutically active amount of a compound according to claim 1.
